(19) United States
(12) Patent Application Publication
(10) Pub. No.: US 2006/0232400 A1
(43) Pub. Date: Oct. 19, 2006

(54) Biological information collecting system

(75) Inventors: Yasunori Ohta, Kanagawa-ken (JP); Takeshi Kamiya, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/384,490

(22) Filed: Mar. 21, 2006

(30) Foreign Application Priority Data
Mar. 23, 2005 (JP) 2005-084790

(51) Int. Cl. A61B 5/00; G08B 1/08; H04Q 7/00

(52) U.S. Cl. 340/539.22; 340/539.23

(57) ABSTRACT

A biological information collecting system has a biological information detecting sensor for measuring and transmitting biological information of a biological subject, the biological information detecting sensor being either disposed in a given environment which the biological subject enters and exits or mounted on the biological subject, an information storage device to be carried by the biological subject, and an entry/exit managing device for detecting the biological subject as it enters or exits the given environment, and acquiring and storing information from the information storage device. The information storage device with ID information of the biological subject being registered therein includes a transmitting and receiving unit for receiving at least the biological information transmitted from the biological information detecting sensor, and transmitting the biological information to the entry/exit managing device and a memory for storing at least the biological information.

### BACKGROUND OF THE INVENTION

1. Field of the Invention

The present invention relates to a biological information collecting system for measuring the biological information of a biological subject placed in a given environment and managing the state of the given environment or the health condition of the biological subject in the given environment based on the measured biological information.

2. Description of the Related Art

Heretofore, there have been known biological information collecting systems as disclosed in Japanese Laid-Open Patent Publication No. 2003-247991, Japanese Laid-Open Patent Publication No. 2003-85292, and Japanese Laid-Open Patent Publication No. 2004-4018.

The biological information collecting system disclosed in Japanese Laid-Open Patent Publication No. 2003-247991 measures the biological information of a biological subject identified by an identifying means and stores the measured biological information together with time information. Based on the frequency distribution of the stored biological information of a plurality of biological subjects, the biological information collecting system confirms a transition of the biological information of an organization that is made up of the biological subjects. The biological information of each of the biological subjects is used statistically only and kept anonymous. Biological subjects may be registered in the identifying means, using magnetic cards, IC cards, or the like.

The biological information collecting systems disclosed in Japanese Laid-Open Patent Publication No. 2003-85292 and Japanese Laid-Open Patent Publication No. 2004-4018 employ a biological information measuring apparatus that is installed in a frequently used room. The biological information measuring apparatus is operated by a person belonging to an organization to measure the biological information of biological subjects of the organization. The biological information collecting systems generate health management data from the measured data and display the generated health management data. The measured biological information may be stored as personal data in an external storage unit, using a personal recognition medium such as a registration card or the like.

However, the conventional biological information collecting systems are not based on a concept for storing biological information in an IC card that is carried by each biological subject.

With the conventional biological information collecting systems, the measured biological information is not recorded in the external storage unit while each biological subject is being conscious of, i.e., is recognizing, the recording of the biological information, but is involuntarily recorded in the external storage unit while each biological subject is not being conscious of, i.e., is not recognizing, the recording of the biological information. Stated otherwise, each biological subject does not have any idea about when its biological information is recorded in the external storage unit. Therefore, each biological subject tends to develop a sense of anxiety in that its biological information may be collected without their permission.

### SUMMARY OF THE INVENTION

It is an object of the present invention to provide a biological information collecting system for recording the biological information of a plurality of biological subjects placed in the same environment, in an external storage unit while each of the biological subjects is well recognizing the recording of the biological information, and managing the state of the environment or the health condition of the biological subjects in the environment, based on the recorded biological information, so that the health condition of the biological subjects in a working environment or a special environment, for example, can be managed while the recording being recognized by the biological subjects.

A biological information collecting system according to the present invention includes a biological information detecting sensor for measuring and transmitting biological information of a biological subject, the biological information detecting sensor being either disposed in a given environment which the biological subject enters and exits or mounted on the biological subject, an information storage device to be carried by the biological subject, and an entry/exit managing device for detecting the biological subject when the biological subject enters or exits the given environment, and acquiring and storing information from the information storage device, the information storage device with ID information of the biological subject being registered therein comprising receiving means for receiving at least the biological information transmitted from the biological information detecting sensor, memory means for storing at least the biological information, and transmitting means for transmitting at least the ID information and the biological information.

With the above arrangement, the biological information of a plurality of biological subjects in the same environment can be recorded in an external storage device while the recording being sufficiently recognized by each of the biological subjects, and the state of the environment or the health condition of the biological subjects in the environment can be managed based on the recorded biological information. Consequently, the health condition of a biological subject or subjects in a working environment or a special environment, for example, can be managed while the recording being recognized by the biological subject or subjects.

The biological information detecting sensor may transmit, in addition to the biological information, date/time data information representing a date/time at which the biological information is measured, the receiving means may receive the date/time data information representing the date/time at which the biological information is measured, in addition to the biological information, and the memory means may further store the date/time data information representing the date/time at which the biological information is measured.

The biological information detecting sensor may transmit, in addition to the biological information, a biological information code for specifying the biological information, the receiving means may receive the biological information code in addition to the biological information, and the memory means may store the biological information in association with at least the biological information code.

The biological information collecting system may further include an environmental information detecting sensor for measuring and transmitting environmental information of the given environment, the receiving means may receive, in addition to the biological information, the environmental information transmitted from the environmental information detecting sensor, and the memory means may further store the environmental information.

The environmental information detecting sensor may transmit, in addition to the environmental information, date/time data information representing a date/time at which the environmental information is measured, the receiving means may receive, in addition to the environmental information, the date/time data information representing the date/time at which the environmental information is measured, and the memory means may further store the date/time data information representing the date/time at which the environmental information is measured.

The environmental information detecting sensor may transmit, in addition to the environmental information, an environmental information code for specifying the environmental information, the receiving means may receive the environmental information code in addition to the environmental information, and the memory means may store the environmental information in association with at least the environmental information code.

The entry/exit managing device may comprise entry/exit detecting means for detecting the biological subject when the biological subject enters or exits the given environment, communicating means for communicating with the information storage device carried by the biological subject to acquire the information stored in the memory means when the entry/exit detecting means detects the biological subject exiting the given environment, and information memory means for storing the information acquired by the communicating means.

With the biological information collecting system according to the present invention, as described above, the biological information of a plurality of biological subjects in the same environment can be recorded in an external storage device while the recording being sufficiently recognized by each of the biological subjects, and the state of the environment or the health condition of the biological subjects in the environment can be managed based on the recorded biological information. Consequently, the health condition of a biological subject or subjects in a working environment or a special environment, for example, can be managed while the recording being recognized by the biological subject or subjects.

The above and other objects, features, and advantages of the present invention will become more apparent from the following description when taken in conjunction with the accompanying drawings in which preferred embodiments of the present invention are shown by way of illustrative example.

### BRIEF DESCRIPTION OF THE DRAWINGS

FIG. 1 is a block diagram of a biological information collecting system according to an embodiment of the present invention;

FIG. 2 is a block diagram of a biological information detecting sensor of the biological information collecting system according to the embodiment;

FIG. 3 is a block diagram of an environmental information detecting sensor of the biological information collecting system according to the embodiment;

FIG. 4 is a plan view of a card-type information storage device;

FIG. 5 is a perspective view of a ring-type information storage device;

FIG. 6 is a perspective view of a pierce-type information storage device;

FIG. 7 is a block diagram of an information storage device of the biological information collecting system according to the embodiment;

FIG. 8 is a diagram showing an example of details of a biological information file;

FIG. 9 is a diagram showing an example of details of an environmental information file;

FIG. 10 is a block diagram of an entry/exit managing device of the biological information collecting system according to the embodiment;

FIG. 11 is a flowchart of a processing sequence of the biological information detecting sensor;

FIG. 12 is a flowchart of a processing sequence of the information storage device; and

FIG. 13 is a flowchart of a processing sequence of the entry/exit managing device.

### DESCRIPTION OF THE PREFERRED EMBODIMENT

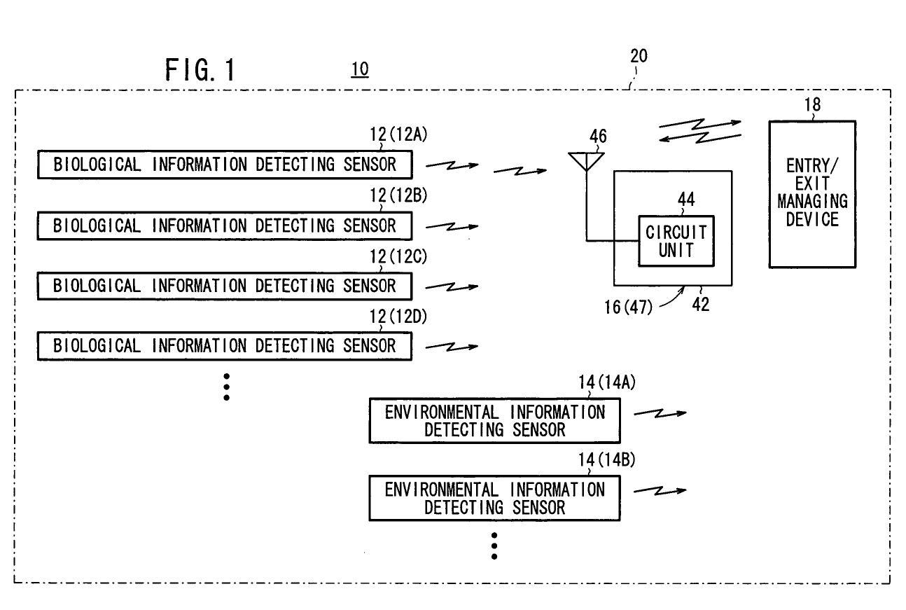

A biological information collecting system according to an embodiment of the present invention will be described below with reference to FIGS. 1 through 13.

As shown in FIG. 1, the biological information collecting system, generally designated by 10, has a plurality of biological information detecting sensors 12, a plurality of environmental information detecting sensors 14, an information storage device 16 that is carried by a biological subject (not shown), and an entry/exit managing device 18.

The biological information detecting sensors 12 are disposed in a given environment 20 that a biological subject enters and exits, and/or mounted on a biological subject placed in the given environment 20. The biological information detecting sensors 12 can measure the biological information of the biological subject and send the measured biological information. The given environment 20 may be a working place where radiation rays, laser beams, chemicals, etc. are handled.

The biological information detecting sensors 12 may comprise an electronic clinical thermometer 12A, an electronic scale 12B, an electronic blood pressure gage 12C, an electronic blood glucose level sensor 12D, etc.

The electronic clinical thermometer 12A measures the body temperature of the biological subject and sends information about the measurement date/time and the measured body temperature, i.e., a measured value, measurement date/time data, and a biological information code representing the body temperature. The electronic scale 12B measures the body weight of the biological subject and sends information about the measurement date/time and the measured body weight, i.e., a measured value, measurement date/time data, and a biological information code representing the body weight. The electronic blood pressure gage 12C measures the blood pressure of the biological subject and sends information about the measurement date/time and the measured blood pressure, i.e., a measured value, measurement date/time data, and a biological information code representing the blood pressure. The electronic blood glucose level sensor 12D measures the blood glucose level of the biological subject and sends information about the measurement date/time and the measured blood glucose level, i.e., a measured value, measurement date/time data, and a biological information code representing the blood glucose level.

The biological information detecting sensors 12 are disposed in the given environment 20 that a biological subject enters and exits, and/or mounted on a biological subject placed in the given environment 20. The biological information detecting sensors 12 may be manually turned on by a switch (not shown) operated by the biological subject to measure necessary data, or may always be worn by the biological subject and may automatically be turned on periodically to measure necessary data.

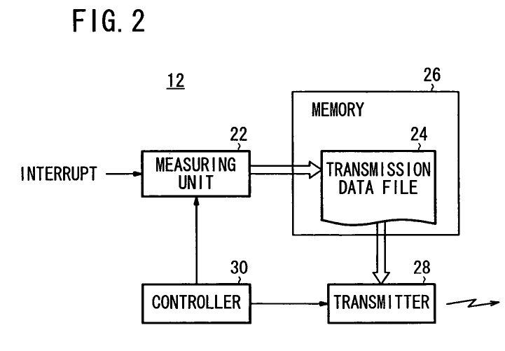

As shown in FIG. 2, each of the biological information detecting sensors 12 basically comprises a measuring unit 22 for measuring data based on an external interrupt activated by the switch or a periodical internal interrupt, a memory 26 having a transmission data file 24 for temporarily storing data measured by the measuring unit 22 (biological information: measured value) and measurement date/time data, a transmitter 28 for sending the data (the biological information and the measurement date/time data) stored in the transmission data file 24 of the memory 26, together with the code specifying the biological information (biological information code), and a controller 30 for controlling the measuring unit 22 and the transmitter 28.

As shown in FIG. 1, the environmental information detecting sensors 14 are disposed in the given environment 20 and/or mounted on the biological subject placed in the given environment 20. The environmental information detecting sensors 14 can measure environmental information in the given environment 20 and send the measured environmental information.

The environmental information detecting sensors 14 may comprise an electronic radiation dose sensor 14A, an electronic gas concentration sensor 14B, etc.

The electronic radiation dose sensor 14A measures a radiation dose in the given environment 20, and sends information about the measurement date/time and the measured radiation dose, i.e., a measured value, measurement date/time data, and an environmental information code representing the radiation dose. The electronic gas concentration sensor 14B measures a gas concentration in the given environment 20, and sends information about the measurement date/time and the measured gas concentration, i.e., a measured value, measurement date/time data, and an environmental information code representing the gas concentration.

The environmental information detecting sensors 14 may be manually turned on by a switch (not shown) operated by the biological subject to measure necessary data, or may automatically be turned on periodically to measure necessary data.

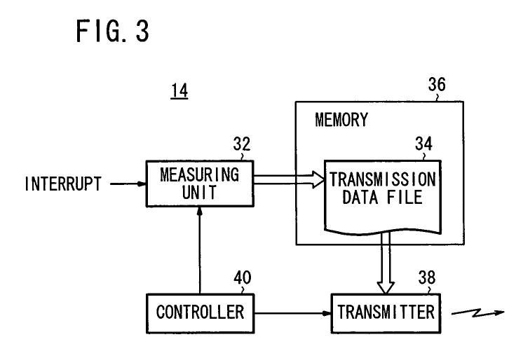

As shown in FIG. 3, each of the environmental information detecting sensors 14 basically comprises a measuring unit 32 for measuring data based on an external interrupt activated by the switch or a periodical internal interrupt, a memory 36 having a transmission data file 34 for temporarily storing the data measured by the measuring unit 32 (environmental information: measured value) and measurement date/time data, a transmitter 38 for sending the data (the environmental information and the measurement date/time data) stored in the transmission data file 34 of the memory 36, together with the code specifying the environmental information (environmental information code), and a controller 40 for controlling the measuring unit 32 and the transmitter 38.

As shown in FIG. 1, the information storage device 16 carried by the biological subject has a wireless IC tag 47 (also referred to as an RFID tag or an electronic tag) in which a circuit unit 44 (including an IC chip or IC chips) and a small-size antenna 46 are mounted integrally. The circuit unit 44 is mounted in a package 42, and the small-size antenna is arranged in the package 42 and connected to the circuit unit 44.

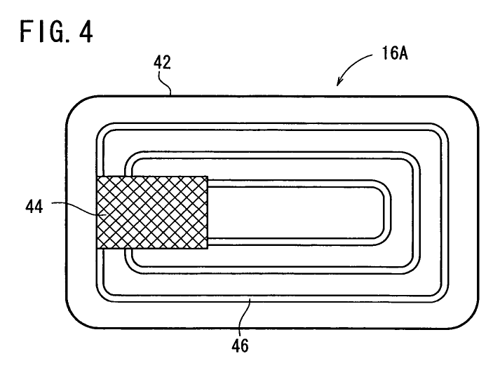

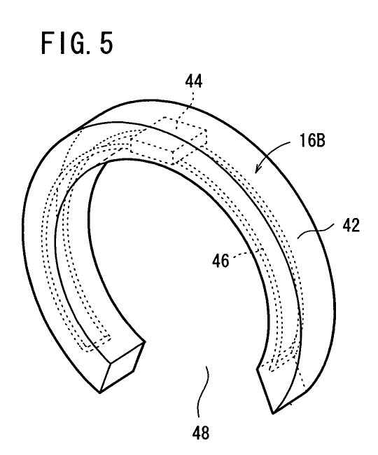

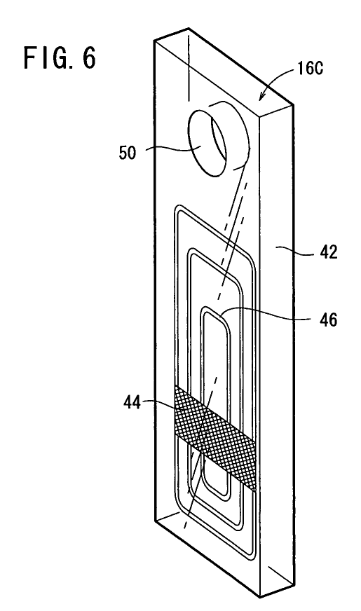

The information storage device 16 may be a card-type information storage device 16A (see FIG. 4), or a ring-type information storage device 16B (see FIG. 5), or a pierce-type information storage device 16C (see FIG. 6) that can easily be carried by the biological subject.

As shown in FIG. 4, the card-type information storage device 16A has a card-shaped package 42 made of synthetic resin and having the size and thickness as a credit card, for example. A circuit unit 44 and a small-size antenna 46 are mounted in the package 42.

As shown in FIG. 5, the ring-type information storage device 16B has a substantially ring-shaped package 42 made of synthetic resin. A circuit unit 44 and a small-size antenna 46 are mounted in the package 42. The package 42 has a cutout 48 defined partly therein to make it wearable on fingers of different diameters of various biological subjects. The package 42 can be put on a thick finger by widening the cutout 48 through elastic deformation or plastic deformation.

As shown in FIG. 6, the pierce-type information storage device 16C has a plate-like package 42 made of synthetic resin and having a through hole 50 defined therein for passage of a wire to be attached to an ear of the biological subject. A circuit unit 44 and a small-size antenna 46 are mounted in the package 42.

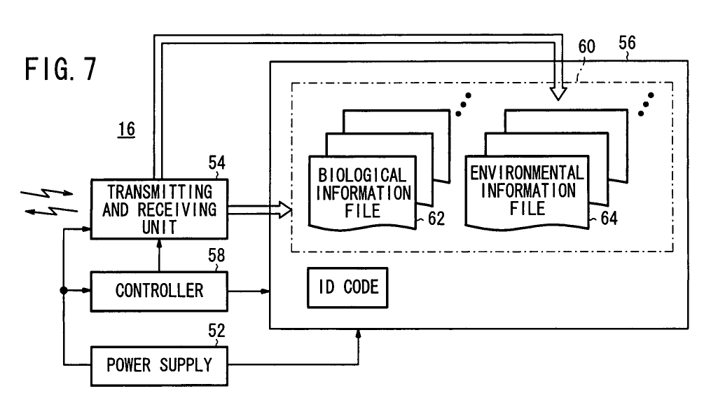

The information storage device 16 has a data transmitting and receiving function and an information storing function. As shown in FIG. 7, the information storage device 16 comprises a power supply 52, a transmitting and receiving unit 54, a memory 56, and a controller 58 for controlling the transmitting and receiving unit 54 and the memory 56.

The transmitting and receiving unit 54 receives information from the biological information detecting sensors 12 and the environmental information detecting sensors 14, and an instruction from the entry/exit managing device 18. Based on the instruction from the entry/exit managing device 18, the transmitting and receiving unit 54 transmits information stored in the memory 56.

The memory 56 stores information that is received from the biological information detecting sensors 12 and the environmental information detecting sensors 14 by the transmitting and receiving unit 54.

The memory 56 also stores ID information (ID codes) at certain addresses and also stores biological subject information 60.

The biological subject information 60 has a plurality of biological information files 62 corresponding to the respective types of biological information codes and a plurality of environmental information files 64 corresponding to the respective types of environmental information codes.

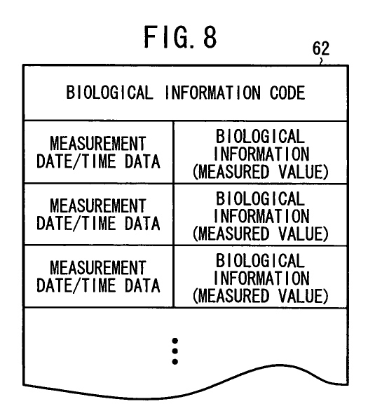

As shown in FIG. 8, each of the biological information files 62 contains measurement date/time data and biological information (measured values) at the measurement dates/times represented by the measurement date/time data, which are related to each other, in addition to the biological information code.

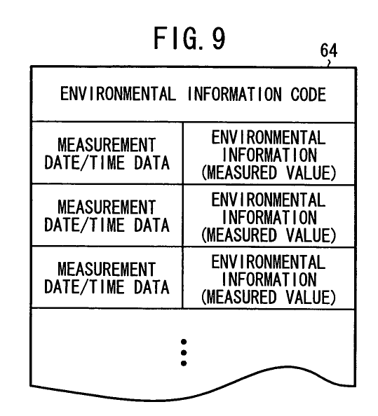

As shown in FIG. 9, each of the environmental information files 64 contains measurement date/time data and environmental information (measured values) at the measurement dates/times represented by the measurement date/time data, which are related to each other, in addition to the environmental information code.

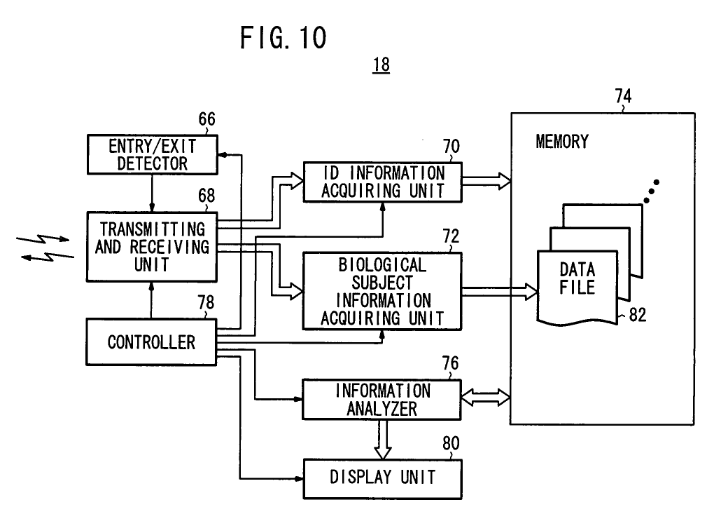

As shown in FIG. 10, the entry/exit managing device 18 comprises an entry/exit detector 66, a transmitting and receiving unit 68, an ID information acquiring unit 70, a biological subject information acquiring unit 72, a memory 74, an information analyzer 76, and a controller 78 for controlling the entry/exit detector 66, the transmitting and receiving unit 68, the ID information acquiring unit 70, the biological subject information acquiring unit 72, the memory 74, and the information analyzer 76. To the entry/exit managing device 18, there is connected a display unit 80 for displaying analytical results from the information analyzer 76, for example.

The entry/exit detector 66 is installed at an entrance/exit of the given environment 20 for detecting the biological subject as it enters and exits the given environment 20. The entry/exit detector 66 may be in the form of an optical sensor or the like.

When the entry/exit detector 66 detects the biological subject as it enters the given environment 20, the ID information acquiring unit 70 instructs, through the transmitting and receiving unit 68, the information storage device 16 carried by the biological subject to send the ID code, and receives the ID code that is sent from the information storage device 16 based on the instruction.

When the entry/exit detector 66 detects the biological subject as it exits the given environment 20, the biological subject information acquiring unit 72 instructs, through the transmitting and receiving unit 68, the information storage device 16 carried by the biological subject to send the biological subject information 60, and receives the biological subject information 60 that is sent from the information storage device 16 based on the instruction.

The memory 74 has a plurality of logically assigned data files 82 corresponding to the respective types of ID codes. Each of the data files 82 stores an ID code and the biological subject information 60 corresponding to the ID code, i.e., a plurality of biological information files 62 corresponding to the types of biological information codes and a plurality of environmental information files 64 corresponding to the types of environmental information codes.

Based on an instruction (including ID information) from the controller 78, the information analyzer 76 reads one of the data files 82 stored in the memory 74 which corresponds to the ID information from the controller 78, and performs a statistical process on data in the data file 82 to analyze the environmental state about the biological subject corresponding to the ID code, i.e., the state of the environment around the biological subject, and the health condition of the biological subject. When all the biological subjects in the given environment 20 have exited the given environment 20, the information analyzer 76 operates based on an instruction from the controller 78 to perform a statistical process on the data contained in the environmental information files 64 in the data files 82 to analyze the overall state of the given environment 20. The analytical results are displayed by the display unit 80 for easy recognition by the biological subjects or administrator.

In FIG. 1, the entry/exit managing device 18 is shown as being installed in the given environment 20. However, the entry/exit managing device 18 may be located in the vicinity of the given environment 20. The entry/exit managing device 18 may be installed in a medical organization for a more professional data analysis to allow the biological subjects and the administrator of the given environment 20 to receive advice from the doctor.

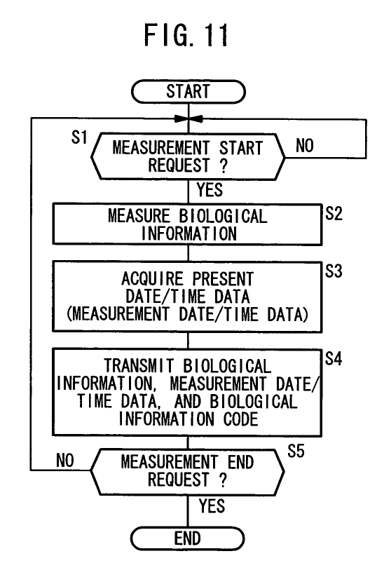

Operation of the biological information collecting system 10 according to the embodiment of the present invention will be described below with reference to FIGS. 11 through 13.

First, operation of each of the biological information detecting sensors 12 will be described below with reference to FIGS. 2 and 11. In step S1 shown in FIG. 11, it is determined whether there is a measurement start request or not based on whether there is an external interrupt activated by the switch (not shown) or a periodical internal interrupt.

If there is a measurement start request, then control goes to step S2 in which the measuring unit 22 (see FIG. 2) starts measuring data, such as the body temperature, the body weight, the blood pressure, or the blood glucose level of the biological subject. The measured value (biological information) is stored in the transmission data file 24 of the memory 26.

When the data measurement is finished, control goes to step S3 in which the measuring unit 22 acquires present date/time data and stores the acquired present date/time data into the transmission data file 24. The measuring unit 22 can acquire the present date/time data easily from a clock (not shown) in the biological information collecting system 10.

Thereafter, in step S4, the transmitter 28 sends the data (the biological information and the measurement date/time data) stored in the transmission data file 24 of the memory 26, together with the biological information code. The data sent from the transmitter 28 is received by the information storage device 16 that is carried by the biological subject who is being currently measured.

In step S5, it is determined whether there is a measurement end request due to a power supply interruption or a maintenance interrupt for the biological information detecting sensor 12 or not. If there is no measurement end request, then control goes back to step S1 to wait for a next measurement cycle. If there is a measurement end request, then the processing sequence of the biological information detecting sensor 12 is put to an end.

Each of the environmental information detecting sensors 14 operates in the same manner as with steps S1 through S5. Therefore, operation of the environmental information detecting sensors 14 will not be described below.

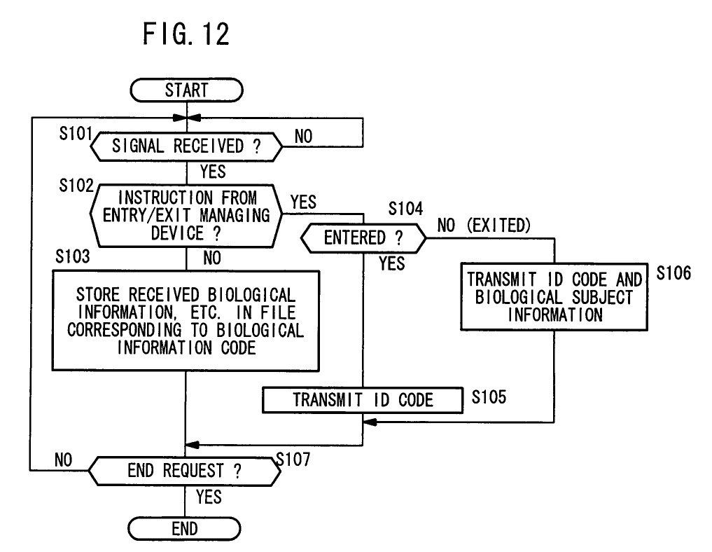

Operation of the information storage device 16 carried by the biological subject will be described below with reference to FIGS. 7 and 12.

In step S101 shown in FIG. 12, the transmitting and receiving unit 54 waits for a signal sent from the biological information detecting sensors 12, the environmental information detecting sensors 14, and the entry/exit managing device 18.

If there is a signal, then control goes to step S102 in which it is determined whether the signal is an instruction from the entry/exit managing device 18 or not.

If the signal is not an instruction from the entry/exit managing device 18, then control goes to step S103 in which the transmitting and receiving unit 54 receives information (a biological information code, biological information, and measurement date/time data) from the biological information detecting sensors 12 or information (an environmental information code, environmental information, and measurement date/time data) from the environmental information detecting sensors 14. The received information is stored in the memory 56. Specifically, if the received information is from the biological information detecting sensors 12, then the measurement date/time data and the biological information are related to each other, and stored in one of the biological information files 62 assigned in the memory 56. The one of the biological information files 62 corresponds to the biological information code in the received information.

If the received information is from the environmental information detecting sensors 14, then the measurement date/time data and the environmental information are related to each other, and stored in one of the environmental information files 64 assigned in the memory 56. The one of the environmental information files 64 corresponds to the environmental information code in the received information.

If the signal is an instruction from the entry/exit managing device 18 in step S102, then control goes to step S104 to determine whether the instruction is an instruction to send the ID code upon the entry of the biological subject into the given environment 20 or not.

If the instruction is an instruction to send the ID code upon the entry of the biological subject into the given environment 20, then control goes to step S105 in which the ID code that is stored in the allocated address in the memory 56 is transmitted through the transmitting and receiving unit 54 to the entry/exit managing device 18.

If the instruction is an instruction to send the biological subject information upon the exit of the biological subject from the given environment 20, then control goes to step S106 in which the ID code and the biological subject information (biological information files 62 and environmental information files 64) stored in the memory 56 are transmitted through the transmitting and receiving unit 54 to the entry/exit managing device 18.

After step S103 or step S105 or step S106, control goes to step S107 to determine whether there is an end request due to a power supply interruption, removal from the biological subject, or a maintenance interrupt for the information storage device 16 or not. If there is no end request, then control goes back to step S101 to wait for a next signal. If there is an end request, then the processing sequence of the information storage device 16 is put to an end.

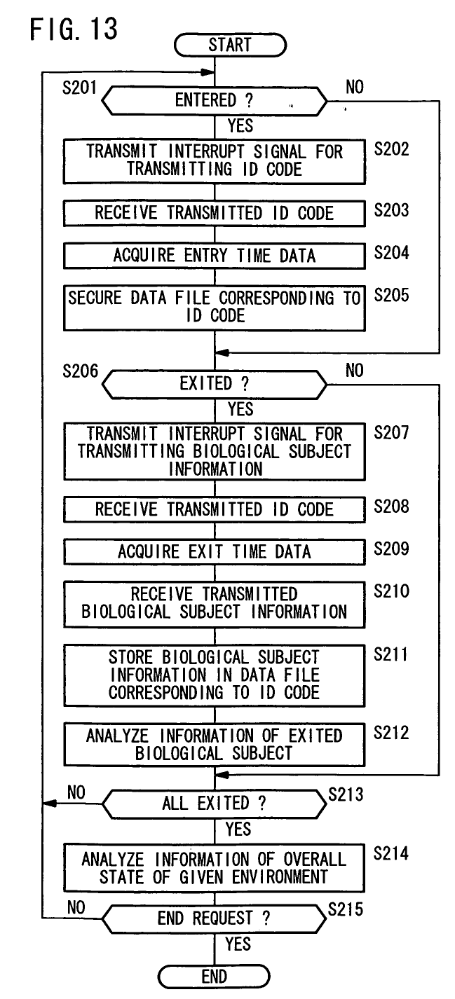

Operation of the entry/exit managing device 18 will be described below with reference to FIGS. 10 and 13. In step S201 shown in FIG. 13, the entry/exit detector 66 determines whether a biological subject has entered the given environment 20 or not. If the entry/exit detector 66 has detected the entry of a biological subject, then control goes to step S202 in which the transmitting and receiving unit 68 transmits an interrupt signal for instructing the information storage device 16 carried by the biological subject to transmit the ID code.

As described above, the instructed information storage device 16 transmits the ID code to the entry/exit managing device 18 in step S105 shown in FIG. 12. In step S203, the ID information acquiring unit 70 of the entry/exit managing device 18 receives the ID code transmitted from the information storage device 16 through the transmitting and receiving unit 68. In step S204, the ID information acquiring unit 70 acquires entry time data. The ID information acquiring unit 70 can acquire the entry time data easily from the clock (not shown) in the biological information collecting system 10.

In step S205, the data file 82 corresponding to the ID code is secured in the memory 74. The data file 82 has a storage capacity large enough to store the biological subject information 60 of the biological subject.

After step S205 or if the entry of a biological subject is not detected in step S201, control goes to step S206 in which the entry/exit detector 66 determines whether a biological subject has exited the given environment 20 or not. If the entry/exit detector 66 has detected the exit of a biological subject, then control goes to step S207 in which the transmitting and receiving unit 68 transmits an interrupt signal for instructing the information storage device 16 carried by the biological subject to transmit the biological subject information 60.

As described above, the instructed information storage device 16 transmits the ID code and the biological subject information 60 to the entry/exit managing device 18 in step S106 shown in FIG. 12. The transmitting and receiving unit 68 of the entry/exit managing device 18 receives the ID code of the information transmitted from the information storage device 16 in step S208 and receives exit time data in step S209. In step S210, the biological subject information acquiring unit 72 acquires the biological subject information 60 of the information transmitted from the information storage device 16. In step S211, the biological subject information 60 is stored in one of the data files 82 which corresponds to the ID code.

In step S212, the information analyzer 76 reads the biological subject information 60 of the biological subject corresponding to the ID code, the entry time data, and the exit time data, and performs a statistical process on the biological subject information 60 to analyze the environmental state about the biological subject corresponding to the ID code, i.e., the state of the environment around the biological subject, and the health condition of the biological subject. The analytical results are displayed on the display unit 80.

In step S213, it is determined whether all the biological subjects that entered the given environment 20 have exited the given environment 20 or not. If not all the biological subjects have exited the given environment 20, then control goes back to step S201 to repeat the processing from step S201. If all the biological subjects have exited the given environment 20, then control goes to step S214. In step S214, the information analyzer 76 reads the respective environmental information files 64 from all the data files 82 stored in the memory 74, and performs a statistical process on the environmental information files 64 to analyze the overall state of the given environment 20. The analytical results are displayed on the display unit 80.

In step S215, it is determined whether there is an end request due to a power supply interruption or a maintenance interrupt for the entry/exit managing device 18 or not. If there is no end request, then control goes back to step S201 to repeat the processing from step S201. If there is an end request, then the processing sequence of the entry/exit managing device 18 is put to an end.

In the biological information collecting system 10 according to the embodiment of the present invention, as described above, the biological information and environmental information of a plurality of biological subjects in the given environment 20 can be recorded in the external storage unit, i.e., the memory 74 of the entry/exit managing device 18 while the recording of the biological and environmental information is being sufficiently recognized by each of the biological subjects. Based on the biological information and environmental information thus recorded, the state of the given environment 20 and the health condition of the biological subjects in the given environment 20 can be managed. Consequently, the health condition of a biological subject or subjects in a working environment or a special environment, for example, can be managed while the recording being recognized by the biological subject or subjects.

In the above embodiment, only one given environment 20 is assumed. However, the state of a plurality of environments and the health condition of biological subjects in such environments may also be managed. Such a modification can easily be realized by managing files using the codes of the different environments as retrieval keys.

Although a certain preferred embodiment of the present invention has been shown and described in detail, it should be understood that various changes and modifications may be made therein without departing from the scope of the appended claims.

What is claimed is:

1. A biological information collecting system comprising:
a biological information detecting sensor for measuring and transmitting biological information of a biological subject, said biological information detecting sensor being either disposed in a given environment which the biological subject enters and exits or mounted on the biological subject;
an information storage device to be carried by the biological subject; and
an entry/exit managing device for detecting the biological subject when the biological subject enters or exits said given environment, and acquiring and storing information from said information storage device;
said information storage device with ID information of said biological subject being registered therein comprising:
receiving means for receiving at least said biological information transmitted from said biological information detecting sensor;
memory means for storing at least said biological information; and
transmitting means for transmitting at least said ID information and said biological information.

2. A biological information collecting system according to claim 1, wherein said biological information detecting sensor transmits, in addition to said biological information, date/time data information representing a date/time at which said biological information is measured;
said receiving means receives said date/time data information representing the date/time at which said biological information is measured, in addition to said biological information; and
said memory means further stores said date/time data information representing the date/time at which said biological information is measured.

3. A biological information collecting system according to claim 1, wherein said biological information detecting sensor transmits, in addition to said biological information, a biological information code for specifying said biological information;
said receiving means receives said biological information code in addition to said biological information; and
said memory means stores said biological information in association with at least said biological information code.

4. A biological information collecting system according to claim 1, further comprising:
an environmental information detecting sensor for measuring and transmitting environmental information of said given environment;
wherein said receiving means receives, in addition to said biological information, said environmental information transmitted from said environmental information detecting sensor; and
said memory means further stores said environmental information.

5. A biological information collecting system according to claim 4, wherein said environmental information detecting sensor transmits, in addition to said environmental information, date/time data information representing a date/time at which said environmental information is measured;
said receiving means receives, in addition to said environmental information, said date/time data information representing the date/time at which said environmental information is measured; and
said memory means further stores said date/time data information representing the date/time at which said environmental information is measured.

6. A biological information collecting system according to claim 4, wherein said environmental information detecting sensor transmits, in addition to said environmental information, an environmental information code for specifying said environmental information;
said receiving means receives said environmental information code in addition to said environmental information; and
said memory means stores said environmental information in association with at least said environmental information code.

7. A biological information collecting system according to claim 1, wherein said entry/exit managing device comprises:
entry/exit detecting means for detecting the biological subject when the biological subject enters or exits said given environment;
communicating means for communicating with said information storage device carried by the biological subject to acquire the information stored in said memory means when said entry/exit detecting means detects the biological subject exiting said given environment; and
information memory means for storing said information acquired by said communicating means.
